(19) United States
(12) Patent Application Publication
(10) Pub. No.: US 2005/0003507 A1
(43) Pub. Date: Jan. 6, 2005

(54) Method for purifying virus

(75) Inventors: Paul Kostel, Richmond, CA (US); Kurt Yanagimachi, Richmond, CA (US); Charles Olson, Moraga, CA (US)

Correspondence Address: ONYX PHARMACEUITICALS, INC., 3031 RESEARCH DRIVE, RICHMOND, CA, 94806, US

(73) Assignee: Onyx Pharmaceuticals, Inc., Richmond, CA (US)

(21) Appl. No.: 10/869,143

(22) Filed: Jun. 16, 2004

Related U.S. Application Data
(60) Provisional application No. 60/479,764, filed on Jun. 18, 2003.

(51) Int. Cl. C12N 7/02

(52) U.S. Cl. 435/239

(57) ABSTRACT

A process for the purification of viruses from a cell lysate preparation is described, consisting of preferably two successive chromatographic steps; the first a clarification step utilizing size exclusion chromatography, and the second, a virus capture and release step using anion exchange chromatography, which successive chromatographic steps have the advantage of purifying virus, and avoiding chromatography buffer conductivity adjustments.

### FIELD

The invention described herein is in the field of virus purification.

### BACKGROUND OF THE INVENTION

There has been resurgence in the use of viruses to treat cancer. See, Lancet Oncology Vol. 3, January 2002, page 17. Perhaps the most studied virus is adenovirus, and McCormick and colleagues have done most of the recent work. They have used an adenovirus with a deletion in the E1B gene which encodes a 55 kd protein that binds to, and inhibits the function of the tumor suppressor, p53. Science, 1996; vol. 274: page 373. The virus targets tumors that lack p53 function, and has entered human clinical trials. Another virus that has been genetically engineered to treat cancer is herpes simplex. Different mutants have been constructed and tested including those with mutations in the ICP34.5 and ICP6 genes. The former encodes the so-called neurovirulence factor, while the later encodes the large subunit of ribonucleotide reductase. The ICP34.5 mutant virus is now in phase I human clinical trials for patients with glioblastoma. Market J, et al., Gene Ther, vol. 2000; vol. 7: page 867.

In addition to adenovirus and herpes simplex, the two most studied oncolytic viruses, considerable work has been, and continues to be conducted on other viruses with oncolytic potential, including vaccinia virus, reovirus, poliovirus, vesiclar stomatitis and newcastle disease virus. Lancet Oncology Vol. 3, January 2002, page 17.

With the renewed interest in viruses as oncolytic agents, or as a means to deliver vaccines or genes, it has become apparent that the methods of cultivating and purifying viruses on the laboratory research scale are not adequate for large scale production that will be required if the viruses are to be used to treat large numbers of patients. Purification of viruses at the research level is generally performed using density-based ultracentrifugation methods. While this method has proven effective for use as a research tool, it is too expensive, time consuming and is not readily scaled up for industrial scale production. A possible alternative to ultracentrifugation is chromatography.

Size exclusion chromatography, alone or in combination with density gradient centrifugation has been used to purify certain plant viruses (Albrechtsen et al., J. Virological Methods 28:245-256, 1990), as well as bovine papilloma virus (Hjorth and Mereno-Lopez, J. Virological Methods 5:151-158 (1982)); and tick-borne encephalitis virus (Crooks et al., J. Chrom. 502:59-68 (1990)). It has also been used for the production of recombinant retroviruses (Mento, S. J., Viagene, Inc., 1994 Williamsburg Bioprocessing Conference).

Haruna et al. in: Virology 13:264-267 (1961)) report using DEAE anion exchange chromatography for purification of types 1, 3 and 8 adenoviruses while Klemperer and Pereir (Virology 9:536-545 1959)) and Philipson (Virology 10:459-465 (1960)) report using the same method with other types of adenoviruses. Also, Blanche F., et al., in: Gene Ther 2000 June; 7(12):1055-62 describe an improved anion-exchange HPLC method for the detection and purification of adenoviral particles.

In addition to size exclusion and anion-exchange chromatography, other chromatographic methods have been used to purify virus. For example, affinity chromatography using monoclonal antibodies (Mab), has been reported to be an effective method for the purification of soybean mosaic virus (Diaco et al., J. Gen. Virol. 67:345-351. 1986). Fowler (J. Virological Methods. 11:59-74. (1985)) used Mab affinity chromatography coupled with density gradient centrifugation to purify Epstein Barr virus.

O'Keeffe R., et al., in: Biotechnol Bioeng 1999 Mar. 5; 62(5):537-45 describe the affinity adsorptive recovery of an infectious herpes simplex virus vaccine using cellufine-sulfate and heparin-HP matrices.

Huyghe et al. (Human Gene Therapy 6: 1403-1416 (1995)) disclose a comparison of several methods for purification of recombinant adenoviruses, including anion-exchange chromatography, size exclusion chromatography, immobilized zinc affinity chromatography, ultracentrifugation, concluding that the preferred process for purification of a recombinant adenovirus is nuclease treatment of a cell lysate, followed by filtration through membrane filters, followed by DEAE chromatography, followed by zinc affinity chromatography.

U.S. Pat. No. 4,724,210 describes a method for purification of influenza virus.

U.S. Pat. No. 4,725,546 describes a method for purification of Japanese encephalitis virus.

U.S. Pat. No. 4,725,547 describes a method for the purification of rabic virus

U.S. Pat. No. 4,855,055 describes the isolation and purification pre-S2 containing hepatitis B virus surface antigen by chemical affinity chromatography.

U.S. Pat. No. 5,602,023 describes a process for preparing a purified virus vaccine comprising the steps of purifying a virus by sucrose gradient ultracentrifugation, rehydration and lyophilization.

U.S. Pat. No. 5,837,520 claims a method for purifying adenovirus consisting of treating a cell lysate which contains viral particles with an enzymatic agent that selectively degrades both unencapsulated DNA and RNA, chromatographing the treated lysate on a first resin, and chromatographing the eluant from the first resin on a second resin, where one resin is an anion exchange resin and the other is an immobilized metal ion affinity resin.

U.S. Pat. No. 6,008,036 describes a method for purifying viruses by chromatography using an anion exchange chromatography step followed by a cation exchange chromatography step, and optionally a metal-binding affinity chromatography step

U.S. Pat. No. 6,194,191 recites a method for producing a purified adenovirus composition comprising growing host cells in a media, providing nutrients to said host cells by perfusion or through a fed-batch process, infecting said host cells with an adenovirus, lysing said host cells to provide a cell lysate comprising adenovirus, wherein said lysis is achieved through autolysis of infected cells, and purifying adenovirus from said lysate to provide a purified adenovirus composition. Various chromaographic steps are also claimed including using anion exchange chromatography.

U.S. Pat. No. 6,261,823 claims a method of purifying adenovirus from a virus preparation, comprising the successive steps of subjecting the virus preparation to anion-exchange chromatography, eluting the adenovirus from the anion-exchange chromatographic medium; and subjecting the anion-exchange eluate to size exclusion chromatography, wherein the adenovirus is eluted from a size exclusion chromatographic medium.

U.S. Pat. No. 6,383,795 claims a method of enriching a solution for an adenovirus using an anion exchange chromatography resin comprising a binding moiety selected from the group consisting of dimethylaminopropyl, dimethylaminobutyl, dimethylaminoisobutyl, and dimethylaminopentyl, such that the adenovirus binds to the chromatography resin. Adenovirus is then eluted from the resin.

U.S. Pat. No. 6,537,793 describes a method of purifying adenovirus comprising contacting a biological medium with a support comprising a cross-linked agarose matrix and ion-exchange groups bound to the cross-linked agarose matrix by a flexible arm, such that contact between the biological medium and the chromatograhpic support separates the viral particles from the biological medium

It is noteworthy that often a central feature of the methods for purifying virus consist of anion-exchange chromatography followed by size exclusion chromatography. Most often there is a filtration step performed prior to the anion-exchange chromatography.

In view of the increasing need for purified oncolytic viruses, or viruses that can be used as viral vectors for gene therapy, improved methods of purification are highly desired.

### SUMMARY OF THE INVENTION

One aspect of the invention is a method for purifying virus from a preparation containing virus using the successive steps of size exclusion chromatography followed by anion exchange chromatography, which successive chromatographic steps have the advantage of clarifying a cell lysate preparation, purifying virus, and avoiding chromatography buffer conductivity adjustments.

Another aspect of the invention is a method of purifying virus from a cell lysate preparation using the successive steps of size exclusion chromatography, and anion exchange chromatography, where the size exclusion chromatography consist of using one or more distinct porous chromatographic materials.

Another aspect of the invention is a method of purifying virus from a cell lysate preparation containing the virus by solubilizing the cell lysate using a detergent prior to the size exclusion chromatography.

Another aspect of the invention is a method of purifying virus from a cell lysate preparation containing the virus by solubilizing the cell lysate using a detergent and a nuclease prior to the size exclusion chromatography.

A feature of the invention is the avoidance of chromatography buffer conductivity adjustments in purifying virus from a cell lysate preparation using size exclusion chromatography followed by anion exchange chromatograhpy.

Yet another feature of the invention is a method for purifying virus involving chromatographys that can be performed sequentially, or in tandem, thereby allowing for rapid purification.

These and other aspects of the invention will become apparent upon a full consideration of the invention presented below.

### BRIEF DESCRIPTION OF THE DRAWINGS

The following drawings demonstrate certain aspects of the present invention, and the invention may be better understood by reference to one or more of these drawings in combination with the detailed description of specific embodiments presented herein.

FIG. 1 shows the overall purification process flow diagram for adenovirus. “XAD-7HP” refers to a Amerlite chromatographic step; “G-50-Fine” refers to the Sephadex G-50 Fine chromatographic step; “AEX” denotes the anion exchange chromatographic step; and UF/DF refers to the ultrafiltration step.

FIG. 2 shows the chromatographic profile of a virus preparation eluate from an Amberlite XAD-7HP/Sephadex™ G-50 Fine column run in tandem. The virus preparation was made from virally infected cells by lysing the cells with Tween™-80 and Benzonase™.

FIG. 3 shows the chromatographic profile of the eluate from a Sephadex G-50 Fine column applied to a Q-Source 30 anion exchange column.

### DETAILED DESCRIPTION OF THE INVENTION

All publications and patent applications cited throughout this patent are incorporated by reference to the same extent as if each individual publication or patent/patent application is specifically and individually indicated to be incorporated by reference in their entirety.

The practice of the invention employs techniques of molecular biology, protein analysis and microbiology, which are within the skilled practitioner of the art. Such techniques are explained fully in, e.g., Current Protocols in Molecular Biology, Ausubel et al., eds., John Wiley & Sons, New York, 1995. Also, the techniques for transfection of the cells, amplification and titration of the adenoviruses have been described previously (F. L. Graham et al., Molecular Biotechnology 3: 207-220, 1995; Crouzet et al., Proc. Natl. Acad. Sci USA 94: 1414-1419, 1997; WO 96/25506).

Modifications and variations of this invention will be apparent to those skilled in the art. The specific embodiments described herein are offered by way of example only, and the invention is not to be construed as limited thereby.

The present invention relates to a process for the purification of virus from a preparation containing virus. By “virus preparation” is intended any solution containing virus, and other materials that the virus is sought to be purified. The virus preparation can be produced by a number of methods, including cultivation in a host cell in vitro including any one of batch, perfusion, or cell factory methods, or in vivo in an appropriate animal host. In the former instance, virally infected cells can be harvested, separated from the growth media, and the virus liberated by lysis of the cells and separation from cellular debris. In the latter instance, a tissue, or organ harboring the virus can be removed and the virus also liberated by lysis of cells that comprise the tissue or organ, and separated from cellular/tissue debris. Virus can also be purified from bodily fluids.

The term “virus” includes wild type, mutant, and recombinant viruses, especially but not exclusively adenovirus, herpes simplex, hepatitis A virus, lentivirus, vaccinia virus, reovirus, poliovirus, mumps, vesiclar stomatitis, parvovirus B19, and newcastle disease virus. A skilled practitioner of this art will appreciate that other viruses may be purified using the process of the instant invention by adapting certain of its features as are appropriate to the virus being purified. The prefered viruses are those readily purified using as a chromatographic step in the purification process, anion exchange chromatography. Such viruses would include adenovirus, lentivirus, which elutes between 0.5-1 M NaCl as a large wide peak, infectious pancreatic necrosis virus, which elutes at a salt concentration between 100 and 125 mM NaCl, hepatitis A virus, and parvovirus B19.

“Lysis” refers to the process of opening virally infected cells by chemical, or physical means, or as part of the viral life cycle thereby allowing for the collection of virus. The latter process is termed autolysis.

By “clarification” is meant the step in the purification of virus from a virus preparation using porous chromatographic material, which step precedes an anion exchange chromatograpic step. Clarification can be achieved in column chromatography or batch format.

By “clarification chromatography” is meant the clarification step in the purification of virus from a virus preparation using porous chromatographic material.

By “porous chromatographic material” is meant viritually any type of material commonly used in the separation of molecules primarily based on their size, and to lesser degrees hydrophobicity and charge. As exemplified herein, “porous chromatographic material” includes dextran (e.g. Sephadex™ resins), or other porous materials that can be composed of a variety of materials including agarose, poly-styrene divinyl-benzene, polymethacrylate, silica, aliphatic acrylic polymers (e.g. Amberlite™ resins), with a variety of surface derivitizations (e.g,, hydrophylic, ionic, hydrophobic, etc.) such that impurities are retained but virus is not.

By “size exclusion chromatography” is meant a method for separating molecules using porous chromatographic material, preferably porous beads. Size exclusion chromatography can consist of one or more distinct types of porous chromatographic material used in a single step, or one or more distinct types of porous chromatographic material used in multiple separate steps, which are conducted prior to anion exchange chromatography. As used herein, an example of “size exclusion chromatography” where more than one porous chromatographic material is used is Amberlite™ XAD7HP and Sephadex™ G-50.

The chromatographys discussed below can be run as individual steps, or sequentially, or in tandem. By “in tandem” is meant that an eluate from one chromatography is directly applied to the next chromatography without an intervening eluate collection step.

### Clarification

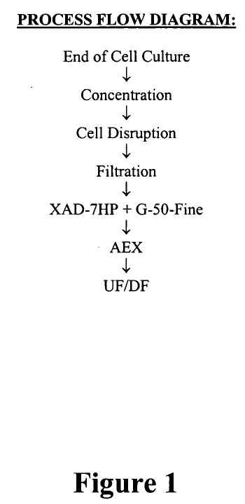

The general purification scheme for purifying virus is shown in FIG. 1. A preferred embodiment of the invention involves clarification using size exclusion chromatography, and preferably consisting of two porous chromatographic materials. The prefered first porous chromatographic material would be composed of non-ionic aliphatic resins, and more preferably such resins could be underivitized non-ionic aliphatic acrylic polymeric resins. More preferred are resins made by Rhom & Hass of the Amberlite series, including Amberlite XAD-7HP. Most preferred is Amberlite XAD-7HP, used in column format, as described in the Examples.

It is important to note, that in purifying virus from certain cell lystates and depending on the amount of cellular aggregates present, it may be desirable to use a single porous chromatographic material to perform size exclusion chromatography, and omit the size exclusion described above. In these instances it may be sufficient to employ a pre-clarification (i.e. filtration) step, discussed below, followed by size exclusion chromatography using a single porous chromatographic material preferably made of dextran, and more preferably certain Sephadex™ resins.

Size exclusion chromatography using Sephadex™ resins, and the anion exchange chromatographic step, discussed below, are well known in the art, and have been described in U.S. Pat. Nos. 5,837,520; 6,194,191; and 6,261,823, although not as successive steps, which is a novel aspect of the instant invention.

Generally, a cell lysate, obtained by means that are well known in the art, and that will be discussed below, is subject to clarification. The eluate containing virus from the size exclusion column is applied to an anion-exchange column. Virus is then eluted from the anion-exchange column. Elution of virus can be monitored by techniques known in the art including optical density, or light scattering. Additionally, the biological properties of the virus prior to and after purification can be determined using well established assays, including plaque assays.

It is important to note again that a novel feature of the instant invention is the use of size exclusion chromatograhpy, followed by anion-exchange chromatography. Previous methods used by skilled practitioners of this art have reversed the order of these two chromatographic steps. See, for example, U.S. Pat. No. 6,261,823.

Without intending to be bound to any particular theory regarding the favorable results obtained by having size exclusion chromatography be used before anion exchange chromatography in the purification process, it is thought that it facilitates virus purification by separating the virus from low molecular weight materials, and much larger molecular weight aggregates consisting of, at least in part, lipid material. This latter property of size exclusion chromatography has hithertofore not been appreciated by skilled practitioners of the art of virus purification. Thus, the inventors have unexpectedly discovered a novel process of purifying virus.

Another feature of the invention that results from the successive use of size exclusion chromatography and anion exchange chromatography is that it greatly reduces or eliminates the need to perform buffer conductivity adjustments between chromatographic steps. As discussed more below, to realize maximum performance of anion exchange chromatography the conductivity of the buffer used to chromatograph virus has, hithertofore, generally required adjustment between purification steps to be in an acceptable range, depending on the type of anion exchanger employed and the charge nature of the virus coat. The need for buffer adjustment prior to anion exchange chromatography is essentially eliminated by the instant invention since the size-exclusion chromatography simultaneously buffer exchanges the virus into the anion exchange equilibration buffer while reducing particulates and lower molecular weight impurities. Thus, the virus eluate can be applied directly to the anion exchange column.

More specifically, as intended herein, size-exclusion chromatography involves separating molecules primarily based on their size, but also based on hydrophobicity and charge using porous chromatographic material, or resins that is preferably an inert gel medium which can be a composite of cross-linked polysaccharides, e.g., cross-linked agarose and/or dextran in the form of spherical beads. The degree of cross-linking determines the size of pores that are present in the swollen gel beads. Molecules greater than a certain size do not enter the gel beads and thus move through the chromatographic bed the fastest. This is true of virus. Smaller molecules, such as detergent, protein, DNA and the like, which enter the gel beads to varying extent depending on their size and shape, are retarded in their passage through the bed. Molecules are thus generally eluted in the order of decreasing molecular size. Viruses, because of their large size, do not enter the pores and generally elute in the void volume. As mentioned above, because molecules that affect buffer conductivity are largely removed, this step yields virus eluate in a buffer that is compatable with anion exchange chromatography without having to alter the conductivity of the buffer. Thus, the virus eluate can be applied directly to the anion exchange column.

Viruses, relative to proteins, are large molecular entities. For example, adenoviruses have a diameter of approximately 80 nm, and thus do not enter the pores of the beads. An additional favorable feature of the use of size exclusion chromatography in the context of virus purification is, as mentioned above, large molecular weight aggregates and particulates consisting of cellular material which would be expected not to enter the beads and thus elute in the void volumn with virus, in fact, are retained, which reduces the rate at which they elute from the column. The result is the unexpected separation of virus from this material. This effect may result from the aggregates/particulates becoming resident for times in the interstitial space between the porous chromatographic material; that is, the space between the porous beads, the size of which is directly proportional to the diameter of the particles in the packed bed, or from an affinity that such aggregates have for the size exclusion chromatographic porous material.

Preferred porous chromatographic resins appropriate for size-exclusion chromatography of viruses are made of dextran, and more preferably are made of cross-linked dextrans. Most preferred are those under the tradename, “SEPHADEX,” available from Amersham Biosciences. The type of SEPHADEX, or other size-exclusion chromatographic resin used is a function of the type of virus sought to be purified, and the nature of the cell culture lysate containing the virus. Sephadex G-50-Fine has a particle size range of 20-80 um and retains and/or retards impurities <30 kD. This combination particle and pore sizes provides good retention of particulates and low molecular weight impurities, and when equilibrated and loaded at <40% (v/v), enables buffer-exchange into the next chromatographic step, anion exchange, without any additional conductivity adjustment. It also permits ready flow through of high molecular weight viruses.

Other size exclusion supports from different materials of construction are also appropriate, for example Toyopearl 55F (polymethacrylate, from Tosoh Bioscience, Montgomery Pa.) and Bio-Gel P-30 Fine (BioRad Laboratories, Hercules, Calif.).

### Anion Exchange Chromatography

The chromatographic step which follows size exclusion chromatography in the invention clarification process of purifying virus is “anion exchange chromatography.” The latter uses a positively-charged organic moiety covalently cross-linked to an inert polymeric backbone. The latter is used as a support for the resin. Representative organic moieties are drawn from primary, secondary, tertiary and quaternary amino groups; such as trimethylaminoethyl (TMAE), diethylaminoethyl (DEAE), dimethylaminoethyl (DMAE), and other groups such as the polyethyleneimine (PEI) that already have, or will have, a formal positive charge within the pH range of approximately 5 to approximately 9.

The support material should be one that is easily derivatizable and possess good mechanical strength. The material can be a natural polymeric substance, a synthetic polymer or co-polymer, or a mixture of natural and synthetics polymers. The support can take the shape of porous or non-porous particles, beads, membranes, disks or sheets. Such supports include silica, hydrophilic polymer (MonoBeads™, Amersham Corporation, Piscataway, N.J.), cross-linked cellulose (e.g. Sephacel™), cross-linked dextran (e.g. Sephadex™) cross-linked agarose (e.g. Sepharose.™), polystyrene, or a co-polymer such as polystyrene-divinylbenzene or one composed of oligoethyleneglycol, glycidylmethacrylate, methacrylate, and pentaerythroldimethacrylate, to which are grafted polymerized chains of acrylamide derivatives.

It is preferred to use an anion exchange resin consisting of DMAE, TMAE, DEAE, or quaternary ammonium groups. A number of anion exchange resins sold under the tradename Fractogel (Novagen) use TMAE, DEAE, DMAE as the positively-charged moiety, and a methacrylate co-polymer background. More preferred are those resins that use quaternary ammonium resins, and most prefered are quaterneary ammonium resins of the type sold under the trade name Q SOURCE-30 (Amersham Biosciences). Q SOURCE-30 has a support made of polystyrene cross-linked with divinylbenzene.

The anion-exchange chromatographic resin, can be used in a traditional (gravity) column chromatography or high pressure liquid chromatography apparatus using radial or axial flow, fluidized bed columns, or in a slurry, that is, batch, method. In the latter method, the resin is separated from the sample by decanting or centrifugation or filtration or a combination of methods. Eluate from the size exclusion column containing virus can be applied directly to the anion exchange resin, and then eluted from this resin by an increasing salt gradient, preferably a gradient, and more preferably a step gradient of sodium chloride.

The principle of ion-exchange chromatography is that charged molecules adsorb to ion exchangers reversibly so that molecules can be bound or eluted by changing the ionic environment. Separation on ion exchangers is usually accomplished in two stages: first, the substance to be separated is bound to the exchanger, using conditions that give stable and tight binding; then the substance is eluted with buffers of different pH, or ionic strength, depending on the properties of the substance being purified.

More specifically, and as applied to the instant invention, the basic principle of ion-exchange chromatography is that the affinity of a virus for the exchanger depends on both the electrical properties of the external coat of the virus, and the relative affinity of other charged substances in the solvent. Hence, bound virus can be eluted by changing the pH, thus altering the charge of the virus, or by adding competing materials, of which salts are but one example. Because different substances have different electrical properties, the conditions for release vary with each bound molecular species. In general, to get good separation, the methods of choice are either continuous ionic strength gradient elution or stepwise elution. For an anion exchanger, either pH is decreased and ionic strength is increased or ionic strength alone is increased. For a cation exchanger, both pH and ionic strength can be increased. The actual choice of the elution procedure is usually a result of trial and error and of considerations of stability of the virus being purified.

It will be appreciated by a skilled practitioner of this art, that the type of anion-exchanger, and the buffers, and salts used to bind and elute the virus will also be a function of the type of virus sought to be purified.

Finally, the eluate from the anion exchange column may be filtrated through a sterilization filter, 0.45 um or smaller made of polyvinylidene fluoride (PVDF), and the filtrate concentrated. The preferred concentration method is ultrafiltration using a polyethersulfon (PES) membrane, and preferably a 500 kD polyethersulfon (PES) membrane. Preferably the filter is of the BioMax cassette series, obtainable from Millipore Corporation, or a hollow-fiber type filter from AG Corporation. Suitable Ultra filtration filters are also available from Sartorius and Pall corporations.

### Cell Lysate Preparation

Virus can be purified from cell lysates prepared from a number of sources, as mentioned above, including cell lines, tissues, bodily fluids, organs, etc. Often virus will be purified from a cell lysate preparation made from virus infected cells, where the cells have been grown using cell culture methods. For example, adenovirus can be isolated from virus-infected cells such as 293 cells, Hela cells, etc. Cells may be infected at high multiplicity of infection (MOI) in order to optimize yield.

Any method suitable for releasing virus from infected cells may be utilized to prepare a cell lysate containing virus. Virus can be released from infected cells using techniques known in the art, or by autolysis. Preferred methods of lysing virally infected cells include using hypotonic solution, hypertonic solution, sonication, pressure, or a detergent. The preferred technique is to use a detergent, and more preferred, depending on the amount of DNA and RNA in the sample, is to also use a nuclease in combination with a detergent.

Numerous detergents are available to solubilize cells, including non-ionic or ionic detergents. The preferred detergents are non-ionic in nature since they tend not to disrupt the structure of the virus, and hence the purified virus maintains its biological activity. Moreover, they have the beneficial property of binding to hydrophobic regions on the external surface of viruses, which regions are associated with undesirable virus aggregation. Thus, these detergents reduce viral aggregation, which increases the efficiency and yield of the purification process.

A widely used class of non-ionic detergents is Tween™. The Tween™ detergents are nondenaturing, nonionic detergents that are composed of polyoxyethylene sorbitan esters of fatty acids. Typically, Tween™ detergents, particularly Tween™ 20 and Tween™ 80, are used as blocking agents to prevent nonspecific binding of proteins to hydrophobic materials such as plastics or nitrocellulose. As applied to the instant invention, this property reduces viral aggregation. Generally, these detergents are used at concentrations of 0.01-1.0%.

The difference between Tween™ 20 and Tween™ 80 is the length of the fatty acid chain. Tween™ 80 is derived from oleic acid with an 18 chain carbon tail, while Tween™ 20 is derived from lauric acid with a 12 carbon chain tail. The longer fatty acid chain makes the Tween™ 80 detergent less hydrophilic than Tween™ 20, but both detergents are soluble in water.

Another class of non-ionic detergents are the Triton™X-detergents. This family of detergents (Triton™X-100, X114 and NP-40) has certain similar basic characteristics, but are different in their specific hydrophobic-hydrophilic nature. These heterogeneous detergents have a branched 8-carbon chain attached to an aromatic ring. This portion of the molecule contributes most of the hydrophobic nature of the detergent. Triton™X-100 and NP-40 are very similar in structure and hydrophobicity and are interchangeable in most applications, including, as applicable to the instant invention, cell lysis.

Another class of non-ionic detergents, Brij™ is similar in structure to Triton™X detergents in that they have varying lengths of polyoxyethylene chains attached to a hydrophobic chain. However, unlike Triton™X detergents, the Brij™ detergents do not have an aromatic ring and the length of the carbon chains can vary. Brij™58 is similar to Triton™X-100 in its hydrophobic/hydrophilic characteristics.

Yet another class of non-ionic detergents consist of octylglucopyranosides and octylthioglucopyranosides. These are nondenaturing, dialyzable, detergents useful for solubilizing cells.

The preferred embodiment detergents for lysing virally infected cells, and purifying viruses there from are Tween-20™, Tween-80™, NP-40™, Brij-58™, Triton X.™-100 or octyl glucoside. More preferred are Tween™-20 and Tween™-80. Most preferred is Tween™-80 used at a concentration of about 1% final (v/v).

An enzymatic agent may be used to treat the cell lysate consisting of one or more enzymes, preferably an RNAse and/or a DNAse, or a mixture of endonucleases as would be known to the ordinarily skilled artisan. It is well known that nucleic acids may adhere to cellular material which can interfere with the invention chromatographic purification scheme by causing cellular or viral aggegation, resulting in little if any virus being recovered. The preferred enzymatic agent for use in this embodiment is Benzonase™, (American International Chemicals), a recombinant non-specific nuclease which rapidly cleaves both RNA and DNA. Other exemplary nucleases include Pulmozyme™ or any other DNase or RNase commonly used in the art.

The ability of Benzonase™ to rapidly hydrolyze nucleic acids makes the enzyme ideal for reducing cell lysate viscosity. Benzonase™ is well suited for reducing the long chain nucleic acid load during purification, thus improving yield.

The most preferred method of making a cell lysate of virally infected cells involves lysing the cells with a detergent, preferably Tween-80™ in the presence of a nuclease, preferably Benzonase.™

Virus can be identified and/or quantified, particularly adenovirus, by any number of techniques known in the art, including anion exchange (AEX) HPLC, similar to that described in Huyghe, et al, Human Gene Therapy 6:1403-1416 (November 1995). At any point after the anion exchange chromatographic step described above virus can also be identified and/or quantified by any number of techniques known in the art, including measuring absorbance, preferably at 260 nm, of a purified fraction, or the observance of virus particles by light scattering, as described in U.S. Pat. Nos. 5,837,520, and 6,316,185, respectively.

Also, the recovery of infectious virus after a particular purification step may be determined by infection of a suitable host cell line. For example, infectious adenovirus may be identified and titrated by plaque assays. Alternatively, infected cells may be stained for the abundant adenoviral hexon protein. Such staining may be performed by fixing the cells with acetone:methanol seven days after infection, and staining with a polyclonal FITC-labeled anti-hexon antibody (Chemicon, Temecula, Calif.). The activity of a purified fraction may be determined by the comparison of infectivity before and after chromatography.

### Pre-Clarification

Prior to the clarification step, the cell lysate preparation following treatment with detergent, or if preferred, detergent and nuclease, may be treated to remove large particulate matter. This can be accomplished by a number of procedures including low speed centrifugation, or filtration. Filtration is preferred, and the type of filter used (i.e. composition and pore size) is within the knowledge of the skilled practitioner of the art to purify a particular virus. Filters made of polypropylene, polysulfone, PVDF, or cellulose acetate can be used. Polypropylene filters are preferred. The preferred porosity of the filter is generally ≦5 um.

The following examples are included to demonstrate preferred embodiments of the invention. A skilled practitioner of the art would, in light of the present disclosure, appreciate that many changes can be made in the specific embodiments and obtain a similar result without departing from the spirit and scope of the invention.

### EXAMPLES

### Example 1

### Purification Of Adenovirus

The general purification scheme is shown in FIG. 1. Virus was monitored at certain steps of the purification process described in this Example. The method consisted of measuring the virus concentration by AEX-HPLC, similar to that described in Huyghe, et al, Human Gene Therapy 6:1403-1416 (November 1995). In summary, a 1 ml Resource-Q column was equilibrated with a buffer containing 300 mM NaCl and 20 mM phosphate buffer, pH 7.5. A linear gradient from 300 mM to 600 mM NaCl was run to elute the virus and the UV absorbance was monitored at 260 and 300 nm. The area of the virus peak was integrated and compared to a cesium chloride purified reference standard.

### Preparation of Cell Lysate

Hela-S3 cells, available from the American Type Culture Collection, Accession No. ATCC CCL-2.2, were infected with adenovirus, Onyx 411, as described by Johnson, et al., in: Cancer Cell. 2002 May; 1(4):325-37. A 70 liter cell culture harvest was concentrated about 3 fold using a 0.65 um hollow fiber filtration system, glycerol was added to 10% final (v/v), and the cells were frozen and held at −70° C. The cells were kept frozen until used. An aliquot of the frozen harvest of about 2,946 grams of material with a volume about 2,860 mls was staged for purification. Just prior to purification of the virus, the cells were placed at 2-8° C. for 12 hours, followed by 30 minutes of thawing the cells in a 37° C. water bath with intermittent mixing until the cells were at 25° C. Next, while mixing the thawed solution vigorously using an impeller and baffled vessel, Tween™-80 and Benzonase™ were added to make a 1% and 100 U/ml final solution, respectively. Approximately 320 mls of a 10% Tween™-80 solution was added, and about 1,272 uls of a solution of 250 U/ul Benzonase™. The final volume was about 3,182 mls. The mixture was incubated at room temperature with stirring at 500 rpm for an additional 90 minutes after which most of the cellular material had solubilized.

The solubilized material was filtered through a pre-washed, Profile II, porosity 5 um, polypropylene filter, Pall No. PCFY1Y050808. This material was then further clarified and buffer exchanged through the Amberlite and G50 columns as described below.

### Clarification

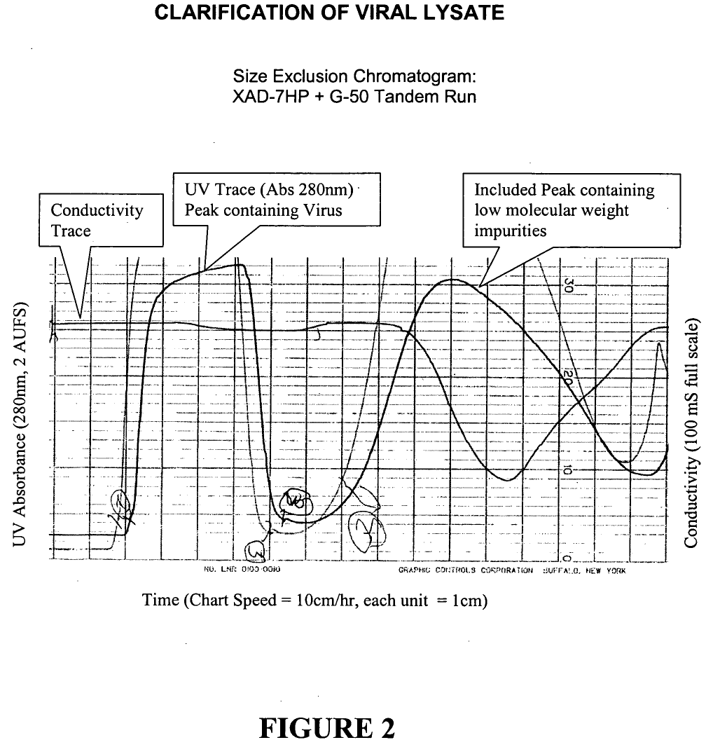

Approximately 2797 mls of the solubilized material prepared as described above was clarified and buffer exchanged by size exclusion chromatography through an Amberlite XAD-7HP column (Rhom & Haas) and a Sephadex G-50 Fine column run in series. The Amberlite column had a diameter of 7 cm, a bed height of 19.5 cm, cross sectional area of 38.5 cm², and a volume of 750 ml. The column was packed by suspending the gel in 1.5-2 volumes of water, causing the resin to have a slurry consistency. Next, the column was sanitized by passing 2 column volumes of 1N NaOH through the column. Prior to loading the cell lysate, the column was washed with 3 column volumes of water, followed by equilibration with anion exchange buffer (AEX) equilibration buffer consisting of 300 mM NaCl, 20 mM Tris (pH 7.5), and 2mM MgCl2. The G-50 column had a diameter of 20 cm, a bed height of 26.8 cm, cross sectional area of 314.2 cm² and a volume of 8420 mls. The resin was prepared by suspending it in 20 times (weight/volume) of a 300 mM NaCl solution containing 2% benzyl alcohol. In order to increase the solubility of benzyl alcohol the NaCl solution was heated to 45-50° C. The G-50 resin was packed as a slurry into the column. Prior to chromatographing the filtered cell lysate, the column was equilibrated with 3 column volumes (CV) of anion exchange buffer (AEX) equilibration buffer. The columns were run connected in tandem at 459 mls per minute. FIG. 2 shows the chromatographic profile. The peak containing virus was pooled by monitoring the UV adsorbance at 280 nm, giving approximately 3608 mls with a virus concentration of 1.5910¹¹ particles/ml.

TABLE 1
Summary of In-Process and Operating Parameters for the tandem
Amberlite XAD-7HP and Sephadex G-50 Fine Chromatography
Amberlite Column size: 7 cm diameter, 19.5 cm bed height,
750 ml bed volume
G-50 Column size: 20 cm diameter, 26.8 cm bed height,
8420 ml bed volume

| Purification Procedure | Parameter | Value |
|---|---|---|
| All Procedures | Temperature | 22degree. C. |
| Equilibration | Flow Rate | 459 ml/min, 3CV/hr |
| | Volume | 3Column Volume, 27.5 L |
| Load | Flow Rate | 459 ml/min, 3CV/hr |
| | Volume | 2797 ml |
| | Concentration | 2.06 × 10¹¹ vp/ml |
| Elution | Flow Rate | 459 mlmin, 3CV/hr |
| | Volume | 3608 ml |
| Fraction Selection | A. sub. 280 | Peak area |

### Anion Exchange Chromatography (AEX)

The anion exchange column, filled with Source 30-Q (Amersham Corporation), had a diameter of 15 cm, a bed height of 15.5 cm, cross sectional area of 176.7 cm² and a column volume of 2,739 mls. As obtained from the manufacturer, the resin comes in a 20% ethanol solution. The resin was resuspended in the ethanol solution and packed into the column. Prior to use, the column was equilbrated with about 8,218 mls of AEX equilibration buffer consisting of 300 mM NaCl, 20 mM Tris (pH 7.5), and 2 mM MgCl2. The equilibration was done at 457 mls per minute for 18 minutes.

Next, the G-50 eluate consisting of 3,608 mls was loaded onto the column through a 0.45 um PVDF Durapore-XL (Millipore Corporation) 10 in capsule filter with a 0.5 um cellulose acetate pre-filter, at a flow rate of 457 ml per minute, followed by washing with equilibration buffer consisting of 2,739 mls of 300 mM NaCl, 20 mM Tris (pH 7.5), and 2 mM MgCl2. Then the column was washed twice; first with a solution containing 100 mM glycine, 20 mM Tris (pH 7.5), 2 mM MgCl2, and second with a solution containing 370 mM NaCl, 20 mM Tris (pH 7.5), and 2 mM MgCl2. Both of these first and second washes consisted of 8,218 mls, and were run at a flow rate of 457 mls per minute for a total of 18 minutes. Next, virus was eluted from the anion exchange resin using an elution buffer consisting of 500 mM NaCl, 20 mM Tris (pH 7.5), and 2 mM MgCl2.

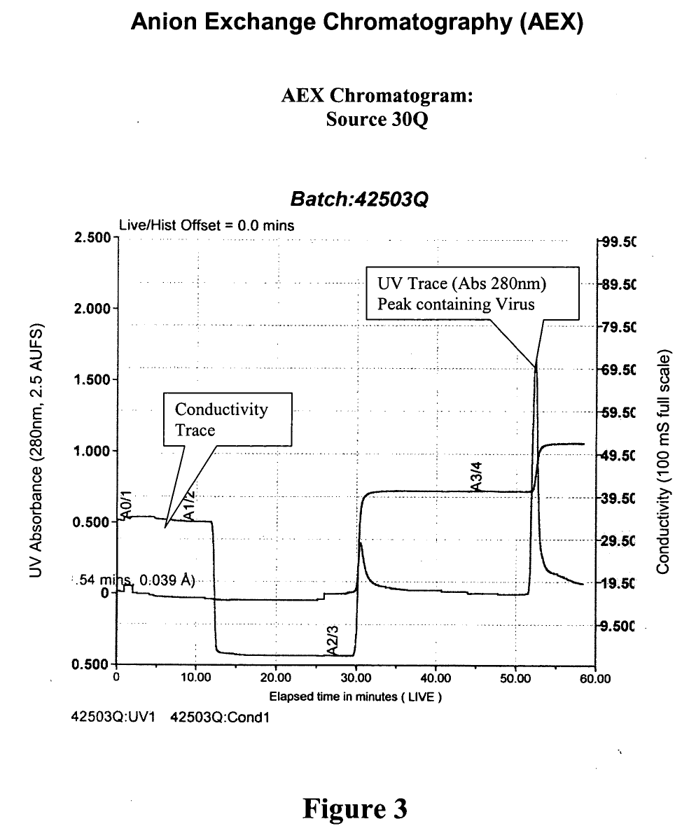

The eluate was pooled by monitoring the UV adsorbance at 280 nm, which yielded a 404 ml pool from the Q Source-30 column with a virus concentration of 7.33×10¹¹ particles/ml. An elution profile is shown in FIG. 3.

A 80% solution (v/v) of glycerol was added to make the pool 10% glycerol, with a final volume of 462 mls. This material was filtered through a 0.45 um Millipore Durapore, PVDF, Millipak-20 filter.

TABLE 3
Summary of In-Process and Operating Parameters for
Q Source-30 Anion Exchange Chromatography
Column size: 15 cm diameter, 15.5 cm bed height,
2,739 ml bed volume

| Purification Procedure | Parameter | Value |
|---|---|---|
| All Procedures | Temperature | ˜22degree. C. |
| Equilibration | Flow Rate | 457 ml/min |
| | Volume | 3 Column Volumes, 8,218 ml |
| Load | Flow Rate | 457 ml/min |
| | Volume | 3,608 ml |
| | Concentration | 1.5910¹¹ vp/ml |
| Equil Wash | Flow Rate | 457 ml/min |
| | Volume | 1 Column Volume, 2,739 ml |
| Wash 1 | Flow Rate | 457 ml/min |
| | Volume | 3 Column Volumes, 8,218 ml |
| Wash 2 | Flow Rate | 457 ml/min |
| | Volume | 3 Column Volumes, 8,218 ml |
| Elution | Flow Rate | 228 ml/min |
| | Volume | 404 ml |
| Fraction Selection | A. sub. 280 | Peak area |
| | Concentration | 7.3310¹¹ vp/ml |
| | | (post glyceration) |

### Concentration by UltraFiltration/Diafiltration

Finally, the eluate obtained from the anion exchange column was concentrated and diafiltered using a Millipore BioMax membrane with a 0.1 sq m 500 kD membrane. The system was run at an inlet pressure of 5 psi and an outlet pressure of 0 psi, with an inlet flow rate of 700 ml/min, which generated a flux rate of 65 ml/min. The glycerated anion exchange eluate was first concentrated ˜2-fold to 1.2×10¹² vp/ml, and then buffer exchanged with 5 diavolumes of the formulation buffer, consisting of 10 mM Tris (pH 8.0), 20 mM NaCl, 10% glycerol and 0.01% Tween-80. This formulated bulk was then held frozen at −70° C.

The invention now being fully described, it will be apparent to one of ordinary skill in the art that many changes and modifications can be made thereto without departing from the spirit or scope of the appended claims.

1. A method of purifying virus from a preparation containing said virus, comprising the successive chromatographic steps of:
a) subjecting the virus preparation to size exclusion chromatography, and eluting the virus from the size exclusion chromatograph; and b) subjecting the eluate of step a) to anion-exchange chromatography, and eluting the virus from said anion-exchange chromatograph.

2. The method of claim 1, wherein the size exclusion chromatography consist of a single porous chromatographic material

3. The method of claim 2, wherein said single porous chromatographic material comprises dextran

4. The method of claim 3, wherein said single porous chromatographic dextran material is Sephadex™.

5. The method of claim 1, wherein the anion-exchange chromatography comprises a chromatographic resin selected from the group consisting of trimethylaminoethyl (TMAE), diethylaminoethyl (DEAE), dimethylaminoethyl (DMAE), or quaterneary ammonium.

6. The method of claim 5, wherein said anion-exchange chromatography comprises the quaternary chromatographic resin Q Source-30.

7. The method of claim 6, wherein said anion-exchange chromatography is followed by a filtration step.

8. The method of claim 7, wherein said filtration step is ultra filtration.

9. The method of claim 1, wherein said virus preparation comprises a cell lysate.

10. The method of claim 9, wherein said cell lysate is made comprising the steps of: infecting cells with a virus; growing said cells in a growth media; and lysing said cells.

11. The method of claim 10, wherein lysing the cells comprises growing said cells for a time sufficient to permit autolysis.

12. The method of claim 10, wherein lysing said cells comprises, isolating the cells from the growth media, and contacting said cells with a detergent at a concentration sufficient to lysis said cells.

13. The method of claim 12, wherein said detergent is a non-ionic detergent.

14. The method of claim 13, wherein said detergent is Tween-80.

15. The method of claim 10, wherein lysing said cells further comprises nuclease treatment of said cell lysate.

16. The method of claim 15, wherein the nuclease comprises a DNAase, an RNAase, or both.

17. The method of claim 16, where said DNAase comprises Benzonase.

18. A method for purifying virus from a cell lysate solution, said method comprising two steps; a first step comprising clarification of said cell lysate solution, wherein said first step yields a solution containing virus that can be further purified in said second step, said second step comprising subjecting said solution obtained from the first step to anion exchange chromatography without adjusting the conductivity of said solution.

19. A method as described in claim 18, wherein said first step comprises subjecting said cell lysate solution to size exclusion chromatography.

20. A method as described in claim 19, wherein said anion exchange chromatography comprises using a chromatographic resin selected from the group consisting of trimethylaminoethyl (TMAE), diethylaminoethyl (DEAE), dimethylaminoethyl (DMAE), or quaterneary ammonium.

21. The method of claim 20, wherein said anion-exchange chromatography comprises the quaternary chromatographic resin Q Source-30.
